(19) United States
(12) Patent Application Publication
(10) Pub. No.: US 2007/0141186 A1
(43) Pub. Date: Jun. 21, 2007

(54) Nopal extract

(76) Inventor: Jong Il Shin, McAllen, TX (US)

Correspondence Address: William F. Ryann, 115 E. Travis St., Ste. 1629, San Antonio, TX, 78205, US

(21) Appl. No.: 11/304,856

(22) Filed: Dec. 15, 2005

(51) Int. Cl. A61K 36/33

(52) U.S. Cl. 424/767

(57) ABSTRACT

A nopal extract isolated from insoluble fibrous waste of a nopal cactus plant. The extract may be concentrated to particularly useful concentrations and provided as a drinkable health supplement. The extract may also be fermented for additional benefit.

### BACKGROUND

Embodiments described relate to dietary supplements for promoting health. In particular, embodiments relate to dietary extracts obtained from the nopal cactus and methods that may be employed to obtain and utilize such extracts.

### BACKGROUND OF THE RELATED ART

Dietary supplements have been used for centuries to promote general health. Many supplements are commercially available in the form of tablets, capsules, or liquids, and their utilization continues to rise. These dietary supplements may include vitamins, minerals, herbs, amino acids and other nutrients. Dietary supplements may often play an important role as part of a healthy diet regimen.

In addition to promoting health generally, particular dietary supplements may be specifically employed to treat certain conditions or ailments. For example, neurotransmitters include amino acids. Therefore, supplements high in amino acid content may be employed to improve memory and mental alertness. Amino acids play a role in helping the body to bum fat and cope with depression. Vitamins, such as B12 and B6 promote metabolic function. The minerals calcium and magnesium specifically promote cardiovascular health.

Whether promoting health generally or targeting specific ailments, dietary supplements or extracts are often derived of natural sources. One such natural source employed for supplements is that of the nopal plant. The nopal plant is a vegetable and may be any of various cacti of the genera Nopalea or Opuntia, including the prickly pear (Opuntia Ficus-Indica) and similar species. These species are good sources of calcium, vitamin B6, and manganese, and other healthful dietary ingredients. Often, the fleshy, oval, edible pad of such a cactus may be diced or blended into a juice or other liquid which is then consumed with the idea being that the drink will include a high amount of vitamins, minerals, and amino acids originating from the nopal pad.

As an alternative to simply dicing the nopal pad for indirect or diluted consumption, as indicated above, efforts have been made to derive the juice itself from the nopal pad for direct consumption without addition of another diluting liquid. Such a ‘single strength’ nopal juice is higher in supplement concentration than the diced or blended nopal pad drink noted above. Regardless, with both the single strength nopal juice and even the diluted nopal pad drink, certain health benefits may be realized from the beneficial dietary ingredients provided by the nopal plant.

Due to the above noted health benefits, efforts have been made to concentrate or enhance the nutritional components of the nopal plant by dehydrating nopal plant material and making it available in a powdered form. The powdered form of the nopal plant may then be compressed into tablets or mixed into another liquid or juice for consumption thereof. Unfortunately, dehydration of nopal plant material into a powdered form substantially removes all of the vitamins, minerals, amino acids, and any other dietary ingredients, leaving behind no more than the fiber. Thus, efforts to enhance or increase the concentration of nopal dietary ingredients have heretofore resulted in a lower concentrate of dietary supplement from that which is already available in a single strength nopal juice. The benefit of a concentrated or enhanced nopal extract remains unrealized.

### SUMMARY

A dietary substance in the form of a nopal extract is described. The nopal extract is concentrated from a nopal plant with fibrous waste of the plant separated therefrom. Remaining nopal liquid extract is concentrated to at least about a 3:1 ratio of water removed to concentrated nopal extract remaining.

### BRIEF DESCRIPTION OF THE DRAWINGS

FIG. 1 is an overview of an embodiment of a nopal liquid extract processing system (NLEPS).

FIG. 2 is an overview of an embodiment of a nopal pad processing mechanism (NPPM).

FIG. 3 is a side cross sectional view of a fermentation tank of the NLEPS, taken from section lines 3-3 of FIG. 1.

FIG. 4 is a perspective view of an embodiment of a bottle filler for coupling to the NLEPS of FIG. 1.

FIG. 5 is a flow chart summarizing an embodiment of processing a nopal liquid extract.

### DETAILED DESCRIPTION

Embodiments are described with reference to certain methods of processing a nopal liquid extract. These may include particular embodiments of concentrating or fermenting a nopal liquid extract. Regardless, embodiments described herein focus on a nopal liquid extract rather than the fibrous solid material of a nopal plant. Therefore, embodiments described herein include the advantage of increased supplement effectiveness.

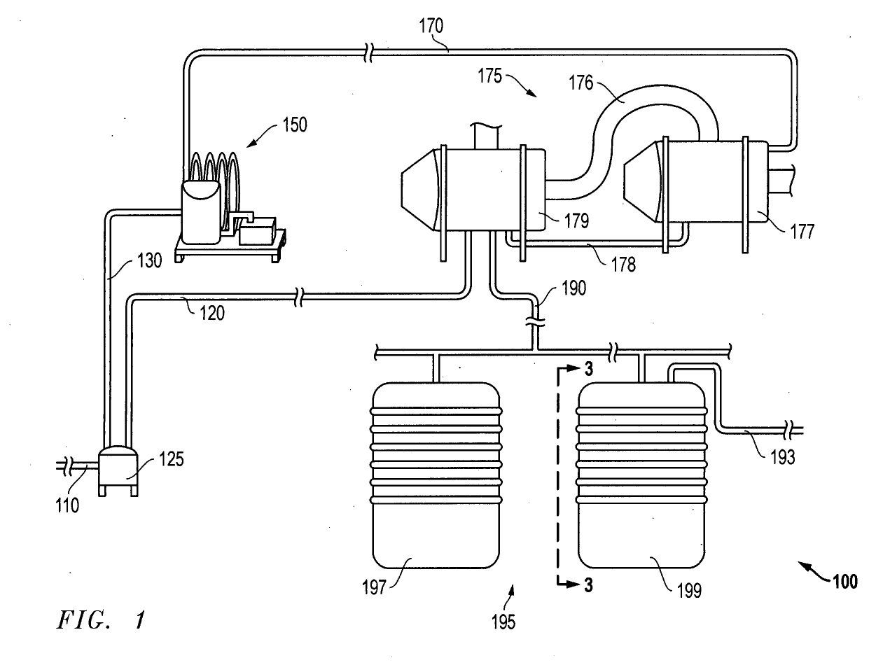

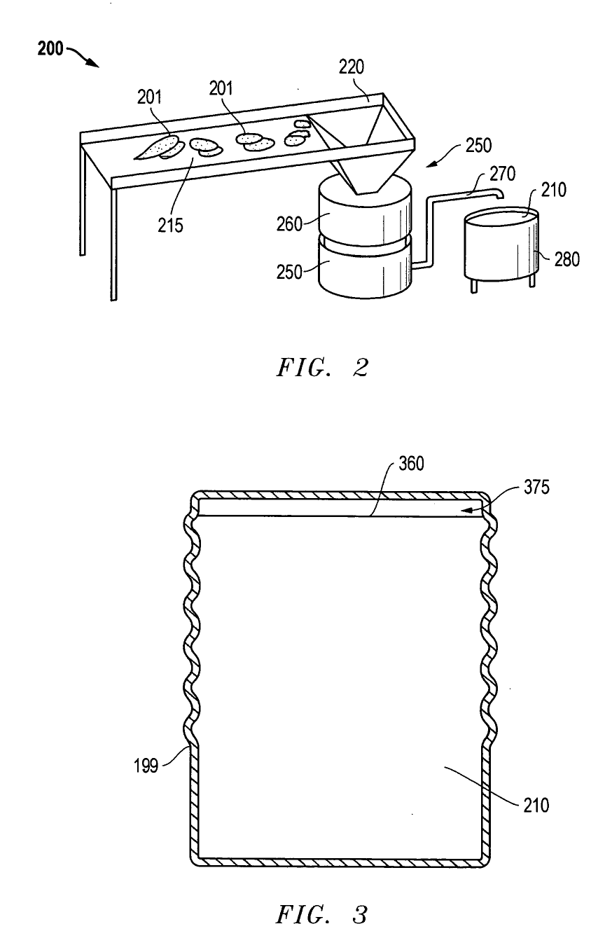

With reference to FIGS. 1 and 2, embodiments of collecting and processing a nopal liquid extract 210 by way of a nopal liquid extract processing system (NLEPS) 100 are described. Nopal liquid extract 210 naturally contains a variety of dietary ingredients including minerals such as Calcium, Copper, Chromium, Iron, Magnesium, Manganese, Phosphorus, and Potassium. Vitamins such as Folate, Pantothenic Acid, Vitamins A, B-1, B-2, B-3, B-6, C, and E are also present along with 18 amino acids and a host of other phytochemicals.

In general terms, a single strength nopal liquid extract 210, described further below, is delivered to a balance tank 125 where it may be directed to an evaporator 175 for concentration of the nopal liquid extract 210. The balance tank 125 may be employed as a safety measure to ensure a constant flow of liquid to the evaporator 175 while the evaporator 175 is running. That is, if a supply of nopal liquid extract 210 is unavailable, the balance tank 125 may pull water from a nearby source (not shown) for advancing through the running evaporator 175. In this manner, damage to the evaporator 175 is avoided.

In addition to the balance tank 125, a pasteurizer 150 may be disposed between the balance tank 125 and the evaporator 175. The evaporator 175 may also lead to a fermentation tank assembly 195 for food process fermentation. Fermented and concentrated nopal liquid extract 210 may be bottled as a drinkable health supplement for consumer use (see FIG. 3).

While, the NLEPS 100 is shown with the balance tank 125 preceding the pasteurizer 150, followed by the evaporator 175 and the fermentation tank assembly 195, the nopal liquid extract 210 may take a variety of application routes. For example, in one embodiment, the nopal liquid extract 210 may pass by the pasteurizer 150 without pasteurization, proceed to the evaporator 175 for concentration, return to the balance tank 125, proceed to the pasteurizer 150 again, this time for pasteurization, then proceed through the NLEPS 100 until the application of fermentation is applied at the fermentation tank assembly 195. A variety of processing options, such as that described here are possible. Additionally, sample ports may be dispersed throughout the lines (120, 130, 170, 178, 190, 193) for testing contamination or taking other readings to help establish the particular application route for the nopal liquid extract 210 through the NLEPS 100.

With particular reference to FIG. 2, a manner of obtaining the nopal liquid extract 210 is shown. The nopal liquid extract 210 is obtained from the nopal pads 201 of the Nopalea or Opuntia, including the common prickly pear cactus. The nopal pads 201 are harvested and washed with water and a conventional food grade sanitizer.

As shown in FIG. 2, the pads 201 may then be placed at a conveyor belt 215 of a nopal pad processing mechanism (NPPM) 200. The conveyor belt 215 advances the nopal pads 201 to a receptacle 220 which directs the nopal pads 201 to a conventional grinder box 260 where the spines and skins of the nopal pads 201 are removed.

Once the nopal pads 201 have been ground as described, nopal liquid extract 210 may be separated from the remaining solid fibrous waste of the nopal pads 201. Cold compression may be employed to help achieve this separation (e.g. see the cold compression chamber 250). As shown in FIG. 2, the nopal liquid extract 210 may then be drawn through an outlet pipe 270 and emptied into a receiving tank 280. Initially the nopal liquid extract 210 is of a gel-like consistency. Therefore, in order to denature and more fully liquefy the nopal liquid extract 210, enzymes may be added to the compression chamber 250 before advancing the nopal liquid extract 210 to the receiving tank 280 shown. These enzymes may be of a cellulase variety. The amount of enzymes added to the nopal liquid extract 210 is negligible in volume. In fact, even with addition of the enzymes, the nopal liquid extract 210 is considered to be of a ‘single strength’ in that it has not been diluted and has yet to be concentrated as described below.

The more fully liquidized single strength nopal liquid extract 210 may then be run through filtration, pasteurization, and any other desired processing before placement in single strength tanks. For example, in one embodiment, a 1 micron to 10 micron filtration may be employed. The single strength nopal liquid extract 210 may now be stored for use as the raw material for processing by the NLEPS 100 as described further below (see FIG. 1).

Continuing with reference to FIGS. 1 and 2, the stored single strength nopal liquid extract 210 may be brought into the NLEPS 100. For example, tanks filled with single strength nopal liquid extract 210 may be coupled to a balance tank 125 in the NLEPS 100 by way of an inlet line 110. The balance tank 125 may serve as the entry point for further processing of the nopal liquid extract 210 as described below.

An initial quantity of single strength nopal liquid extract 210 may be advanced from the balance tank 125 to a pasteurizer 150 through a pasteurization line 130. The nopal liquid extract 210 may be pasteurized at the pasteurizer 150 by heating to a temperature of between about 150° F. and 230° F., preferably about 190° F. Pasteurization may also take place as a later application. Single strength nopal liquid extract 210 may then be advanced to an evaporator 175 where it may be concentrated as described further below. Once concentrated to the desired strength, the nopal liquid may be returned to the balance tank 125 by way of a return line 120. In one embodiment, bacterial content or final concentration of the nopal liquid extract 210 may be monitored by sample ports throughout the NLEPS 100 to help establish and redirect further pasteurization or concentration as needed.

As indicated above, the evaporator 175 may be employed to take the nopal liquid extract 210 from a single strength to a concentrated strength. That is, upon delivery to the evaporator 175, the nopal liquid extract 210 consists of water and dietary ingredients or constituents such as certain vitamins, minerals, and amino acids as noted above. Upon delivery to the evaporator 175, the nopal liquid extract 210 is of a certain ‘single’ strength or concentration of dietary ingredients in water. In fact, the vast majority of the nopal liquid extract 210 at this point is water (i.e. perhaps about 95%). Therefore, the evaporator 175 may be employed to remove much of this water and concentrate the nopal liquid extract 210 with respect to its dietary ingredients.

For example, in one embodiment, the nopal liquid extract 210 is concentrated to seven to one (7:1) ratio by the evaporator 175. More specifically, in one embodiment, 21,000 gallons of single strength nopal liquid extract 210 may be provided to the evaporator 175 where 18,000 gallons of water are removed from the nopal liquid extract 210, leaving 3,000 gallons of concentrated nopal liquid extract 210. In another embodiment the evaporator 175 may be employed to provide 2,095 gallons of concentrated nopal liquid extract 210 from 22,000 gallons of single strength nopal liquid extract 210, a 10.5:1 concentration.

Embodiments of concentrated nopal liquid extract 210 may be concentrated anywhere from about 3:1 to about 65:1 or more by the evaporator 175 as described above. In one embodiment the nopal liquid extract 210 may even be concentrated to between about 18:1 and about 65:1 or more for the formation of a solid nopal supplement of nopal constituents, for example, to be taken in pill form. Concentrations of at least about 3:1 result in a concentrated nopal liquid extract 210 displaying the unique health benefits described below. In fact, at a concentration of about 7:1 as noted above, one ounce of nopal liquid extract 210 qualifies under Food and Drug Adminstration (FDA) guidelines as a “Good Source” of Calcium, Thiamin, Riboflavin and Chromium, as “High in” Vitamin B6, Folate, and Pantothenic Acid, and a “High Potency Source” of Manganese.

As indicated above, concentrations of the nopal liquid extract 210 that are at or above about 3:1 provide particularly unique health benefits. In fact, preliminary tests indicate that such concentrations may be particularly effective in treating arthritic pain, stomach ulcers and elevated cholesterol and blood sugar levels. Generalized immune and digestive system health advantages may also be realized. Furthermore, concentrations of the nopal liquid extract 210 at or above about 3:1 as described herein substantially maintain their inherent phytochemical ratios. That is, while concentrated by removing water, the phytochemical ingredients of the nopal liquid extract 210 are retained in substantially the same ratio with respect to one another as were present prior to concentration. This helps ensure that any beneficial synergistic interplay between the different dietary ingredients (i.e. phytochemicals) of the nopal liquid extract 210 is retained.

Referring now to the evaporator 175 shown in FIG. 1, first 177 and second 179 evaporator tanks are shown. However, the evaporator 175 may include any number of evaporator tanks. For example, in one embodiment, the evaporator 175 includes 4 tanks. In this embodiment, the first evaporator tank 177 with nopal liquid extract 210 therein may be heated such that water vapor rises from the nopal liquid extract 210 and is directed to a vapor tube 176 leaving an initial concentrate of nopal liquid extract 210. This initial concentrate of nopal liquid extract 210 may then be advanced via a tank line 178 to the second evaporator tank 179 where it may be further concentrated with additional evaporation. In the embodiment shown, water vapor from the vapor tube 176 is used to help heat the second evaporator tank 179 in a manner that does not reintroduce the vapor to the concentrated nopal liquid extract 210 in the second tank 179. Following evaporation in the second tank 179, the nopal liquid extract 210 may similarly be advanced to additional evaporative tanks, such as the third and fourth of the embodiment described here. This process may continue until the desired concentration is attained.

The degree of concentration of the nopal liquid extract 210 as described above is a function of the rate of flow of nopal liquid extract 210 through the evaporator 175. That is, the longer it takes the nopal liquid extract 210 to pass through the entire evaporator 175, the greater the evaporation of water from the nopal liquid extract 210 achieved. Thus, user defined parameters may be set, calibrated, and reset to determine exactly how and to what extent the evaporative process is to proceed. Regardless, embodiments described herein focus on achieving a uniquely beneficial degree and level of concentration.

Once concentrated, the nopal liquid extract 210 may be subjected to an additional 1 micron to 10 micron filtration. Concentration of the nopal liquid extract 210 to the desired degree may be analyzed and confirmed by sample ports as described above. Nevertheless, if further concentration is required, the nopal liquid extract 210 may be redirected through the evaporation process as noted above. Alternatively, properly concentrated nopal liquid extract 210 may be directed through the system without employing evaporative techniques.

With the desired concentration and other properties achieved, the nopal liquid extract 210 may be further processed before fermentation. For example, in one embodiment, the nopal liquid extract 210 may be directed to mixing tanks to have preservatives and/or other constituents, such as Stevia, added prior to additional pasteurization and subsequent fermentation. Preservatives that may be employed include Potassium Sorbate, Sodium Benzoate, grapefruit seed extract, citric acid, and other agents. In one embodiment, enzymes may be added to help ensure food process fermentation as described below.

Continuing now with reference to FIGS. 1 and 3, the concentrated nopal liquid extract 210 is eventually directed via a fermentation line 190 to fermentation tanks 197, 199 for food process fermentation. In the embodiment of FIG. 1, two fermentation tanks 197, 199 are shown. However, a multitude of such tanks may be employed. In one embodiment, each tank 197, 199 is filled at different times. For example, at a given point in time, a first tank 197 may have been filled and aging or fermenting for a period of 15 days while a second tank 199 has been filled and aging or fermenting for a period of 30 days. Any number of fermentation tanks 197, 199 may be employed in this manner, each holding nopal liquid extract 210 of a different age. In fact, the traditional benefits of food fermentation, such as preservation and enrichment may be maximized by aging the nopal liquid extract 210 for a period of between about 15 days and about 2,200 days.

Referring specifically to FIG. 3, a side cross sectional view of the second fermentation tank 199 of the NLEPS 100 is shown, taken from section lines 3-3 of FIG. 1. Headspace 375 is shown where air is allowed above the aging nopal liquid extract 210. An air-nopal interface 350 is shown where components of the air in the headspace 375, such as oxygen are able to interact with the nopal liquid extract 210 in furthering the fermentation process.

Continuing with reference to FIGS. I and 3, the nopal liquid extract 210 may be reevaluated following fermentation at the fermentation tanks 197, 199. This allows for re-pasteurization at the pasteurizer 150 as following fermentation of the nopal liquid extract 210 and before bottling thereof as described below. That is, if, based on the reevaluation of the nopal liquid 125, additional pasteurization is desired, the nopal liquid 125 may be advanced through the NLEPS 100 but without added evaporation at the evaporator 175.

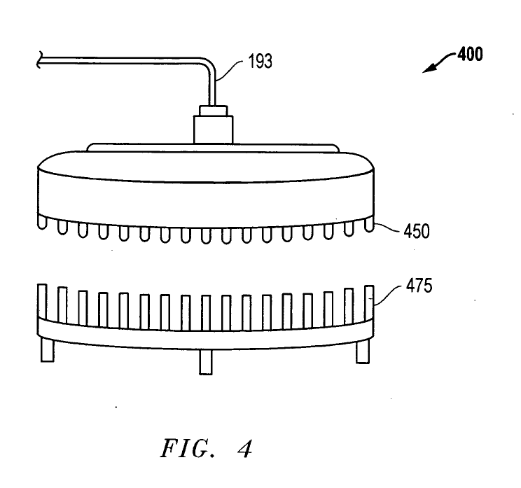

Referring now to FIGS. 1 and 4, a bottle filler 400 is shown. A safe, concentrated, and fermented supply of nopal liquid extract 210 may be supplied to the bottle filler via bottle line 193. The bottle filler 400 is shown with valves 450 coupled to the bottle line 193 and adjacently above bottle supports 475. In the embodiment shown, the bottle filler 400 is rotable and may be positioned adjacent to a conveyor belt for delivering sorted empty bottles. In one embodiment the bottle filler 400 may, while rotating, sequentially secure bottles between the valves 450 and bottle supports 475, fill the bottles with nopal liquid extract 210, and release filled bottles for capping, casing and any further processing. Thus, concentrated and fermented nopal liquid extract 210 may be available for shipping and ultimate consumer consumption.

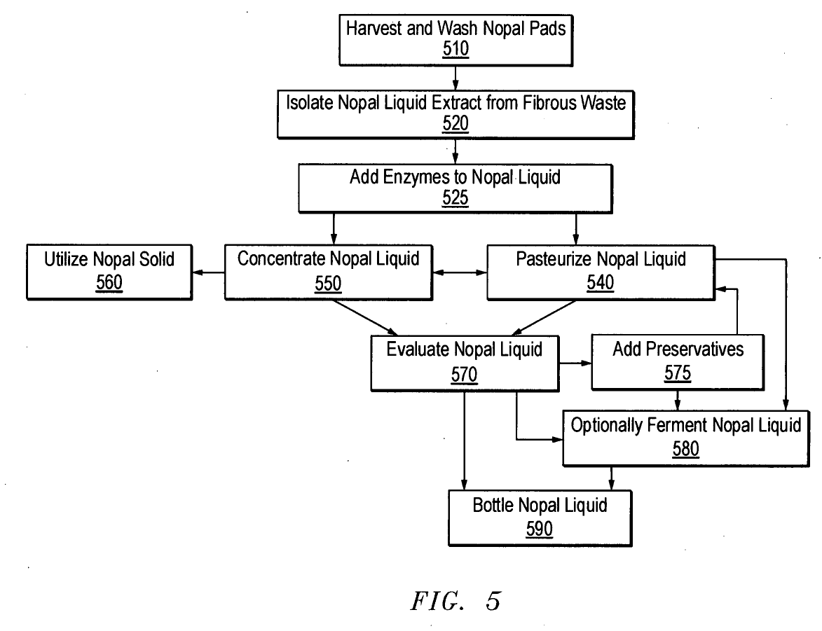

Referring now to the flow-chart of FIG. 5, and with additional reference to FIGS. 1-4, a method of obtaining a nopal liquid supplement in a concentrated and/or fermented form is described. With added emphasis on FIGS. 2 and 5 in particular, initially, nopal pads 201 are harvested and washed as indicated at 510. A grinder box 260 and cold compression chamber 250 are employed to help separate spines, skin, and other fibrous waste from the nopal liquid extract 210 of the nopal pads 201. That is, the nopal liquid extract 210 is isolated as indicated at 520. As indicated at 525, enzymes may then be added to the nopal liquid extract 210. The nopal liquid extract 210 may then optionally be transported to the NLEPS 100 of FIG. 1 for processing.

With added emphasis on FIGS. 1 and 5 in particular, the nopal liquid extract 210 may be circulated through the NLEPS 100 between the pasteurizer 150 and the evaporator 175 for concentration 550 and pasteurization 540 along with possible further analysis or evaluation 570. Although, in one embodiment further analysis may be forgone and the nopal liquid extract 210 solidified for use as indicated at 560. Where evaluation 570 dictates, the nopal liquid extract 210 may be further pasteurized 540 or concentrated 550. Alternatively, however, the nopal liquid extract 210 may be ready for fermenting 580 and bottling 590. However, fermenting 580 is not necessarily required prior to bottling 590. Additionally, preservatives or other constituents may be added to the nopal liquid extract 210 as indicated at 590 (followed by further pasteurization 540).

Embodiments above describe forms of nopal extract which are obtained by methods which allow for retention of substantially all of the vitamins, minerals, amino acids, and other dietary ingredient content. This is achieved in a manner that provides an increased concentration of nopal dietary ingredients not previously available which may be enhanced by fermentation and have particular effectiveness in treating certain ailments. Although exemplary embodiments are described with reference to particular concentration methods and a nopal extract in liquid form, additional embodiments are possible. For example, the nopal liquid may be concentrated to a variety of concentrations not specifically mentioned herein, including concentrates of a solid form. Additionally, enhancement of the nopal supplement by fermentation or other additional measures may be optionally employed. Furthermore, many changes, modifications, and substitutions may be made without departing from the spirit and scope of the described embodiments.

I claim:

1. A dietary substance comprising a concentrated nopal extract from a nopal plant, the nopal plant having waste removed therefrom to leave a single strength nopal liquid, the single strength nopal liquid concentrated by removing water therefrom to at least about a 3:1 ratio of water removed to said concentrated nopal extract remaining.

2. The dietary substance of claim 1 wherein the concentrated nopal extract includes one of calcium, chromium, iron, magnesium, manganese, phosphorus, potassium, vitamin A, vitamin B-1, vitamin B-2, vitamin B-3, vitamin B-6, vitamin C, vitamin E, Folate, Pantothenic acid, and an amino acid.

3. The dietary substance of claim 1 wherein the concentrated nopal extract is for treating one of arthritic pain, stomach ulcers, an elevated cholesterol level, and an elevated blood sugar level.

4. A dietary substance comprising a concentrated nopal liquid extract from a nopal plant, the nopal plant having waste removed therefrom to leave a single strength nopal liquid, the single strength nopal liquid concentrated by removing water therefrom to at least about a 3:1 ratio of water removed to said concentrated nopal liquid extract remaining, said concentrated nopal liquid extract for bottling as a drinkable health supplement.

5. The dietary substance of claim 4 wherein a flavoring agent is added to said concentrated nopal liquid.

6. The dietary substance of claim 4 wherein a preservative is added to said concentrated nopal liquid selected from a group consisting of potassium sorbate, sodium benzoate, grapefruit seed extract, and citric acid.

7. The dietary substance of claim 4 wherein said concentrated nopal liquid is stored for fermentation for at least about fifteen days.

8. The dietary substance of claim 7 wherein an enzyme is added to said concentrated nopal liquid to enhance the fermentation.

9. A dietary substance comprising a concentrated nopal extract from a nopal plant, the nopal plant having waste removed therefrom to leave a single strength nopal liquid, the single strength nopal liquid concentrated to a solid of nopal constituents to provide said concentrated nopal extract.

10. The dietary substance of claim 9 wherein the single strength nopal liquid is concentrated by removing water therefrom to beyond about an 18:1 ratio of water removed to said concentrated nopal extract.

11. A method of preparing a concentrated nopal extract comprising:
isolating a single strength nopal liquid from a nopal plant; and
concentrating the single strength nopal liquid by removing water therefrom to at least about a 3:1 ratio of water removed to the concentrated nopal extract remaining.

12. The method of claim 11 further comprising adding an enzyme to the single strength nopal liquid following said isolating.

13. The method of claim 11 wherein said concentrating is to beyond about an 18:1 ratio of water removed to the concentrated nopal extract remaining, the concentrated nopal extract remaining being a solid.

14. The method of claim 11 wherein said concentrating is achieved with a multiple stage evaporator.

15. The method of claim 11 further comprising pasteurizing the single strength nopal liquid at a temperature of between about 150° F. and about 230° F.

16. The method of claim 15 further comprising evaluating a portion of the concentrated nopal extract for additional pasteurizing.

17. The method of claim 11 wherein the concentrated nopal extract is a liquid extract for bottling as a drinkable health supplement.

18. The method of claim 17 further comprising adding a preservative to the concentrated nopal extract prior to the bottling.

19. The method of claim 17 wherein the bottling is achieved with a rotable bottle filler.

20. The method of claim 17 further comprising evaluating a portion of the concentrated nopal extract for additional concentrating.

21. A method of preparing a concentrated nopal extract comprising:
isolating a single strength nopal liquid from a nopal plant;
concentrating the single strength nopal liquid by removing water therefrom to at least about a 3:1 ratio of water removed to the concentrated nopal extract remaining; and
fermenting the concentrated nopal extract for at least about 15 days.

22. The method of claim 21 further comprising adding one of a preservative and an enzyme prior to said fermenting.
